(19) United States
(12) Patent Application Publication
(10) Pub. No.: US 2009/0300830 A1
(43) Pub. Date: Dec. 10, 2009

(54) Goggle with removable lens

(76) Inventor: Jerome J.M. Mage, Los Angeles, CA (US)

Correspondence Address: STETINA BRUNDA GARRED & BRUCKER, 75 ENTERPRISE, SUITE 250, ALISO VIEJO, CA, 92656, US

(21) Appl. No.: 12/156,845

(22) Filed: Jun. 5, 2008

(51) Int. Cl. A61F 9/02

(52) U.S. Cl. 2/441; 2/426; 2/452

(57) ABSTRACT

A sports goggle having a lens removably attachable to a frame is disclosed. The lens may be secured to the frame via a frame clip. The frame clip may be secured to the frame via a bayonet wherein aligned apertures of the frame clip and frame have an oblong configuration sized and configured to receive an oblong enlarged head of a pin of the bayonet when the bayonet is in a release position. To secure the frame clip to the frame, the pin is inserted into the aligned apertures and the bayonet is rotated to a worn position which misaligns the oblong enlarged head of the pin and the oblong configured aligned apertures. Moreover, the sports goggle increases comfort compared to prior art sports goggles. In particular, the lateral distal portions of the frame are fabricated from a soft compound or material embedded onto a harder compound or material of the frame.

### CROSS-REFERENCE TO RELATED APPLICATIONS

Not Applicable

### STATEMENT RE

### FEDERALLY SPONSORED RESEARCH/DEVELOPMENT

Not Applicable

### BACKGROUND

The present invention relates to a sports goggle with a removably attachable lens.

Various sports utilize goggles to prevent debris (e.g., snow, dirt, rock, etc.) from entering the eyes of the participant. By way of example and limitation, snowboarders may have a snow goggle strapped around his/her face or head. The snow goggle has a frame which holds a transparent lens through which the snowboarder may view his/her surroundings/environment and by which snowflakes or snow are prevented from entering into the snowboarder's eyes.

During use, snowflakes may melt on the lens of the snow goggle. The snowboarder may wipe the melted water off of the lens with a rubber squeegee built into the gloves of the snowboarder. Over a period of time, the exterior surface of lens may be scratched to an extent that the snowboarder is not able to clearly see his/her environment. The source of the scratches may be the repeated wiping of the melted snowflake from the exterior surface of the lens with the rubber squeegee. Another source of scratches is that the sports goggles may be stored without a cover. In this instance, the lens of the goggle may be placed on a tabletop or within a bag. Contents within the bag and the tabletop surface may itself scratch the exterior surface of the lens.

Another deficiency, in prior art sports goggles, is the discomfort in wearing the sports goggle. The sports goggle is uncomfortable to wear due to the manner in which the sports goggle is secured to the face of the wearer. In particular, the sports goggle may comprise an elastic head strap. The lens and frame of the sports goggle are disposed in front of the wearer's face while the head strap is routed to the rear of the wearer's head. The head strap is tensioned such that the frame is pressed against the wearer's face. The head strap is attached to the lateral sides of the frame. The tension of the head strap pulls on the lateral sides of the frame and presses the sports goggle unevenly against the wearer's face. The uneven pressure against the wearer's face is one source of discomfort.

Accordingly, there is a need in the art for an improved sports goggle.

### BRIEF SUMMARY

The sports goggle discussed herein addresses the deficiencies in the prior art discussed above, discussed below and those that are known in the art.

The sports goggle may have a lens that is removably attachable to a frame. This permits the user to service (e.g., clean) the lens or replace the lens with a fresh, clean lens. In particular, the upper and lower middle portions of the frame may have lens channels sized and configured to receive the upper and lower peripheral edges of the lens. This holds the lens in position with the frame. Nonetheless, the lens may still be removed from the frame due to the bendability of the frame. As such, the lens may be secured to the frame via left and right frame clips. The left and right frame clips may be disposed over the lateral distal portions of the lens and secured to the frame to sandwich the lens between the frame clips and the frame. When the frame clips are secured to the frame, the lens is also secured to the frame. The lens will not dislodge from the frame during normal use of the sports goggle. When the frame clips are removed from the frame, the lens is removable from the lens channel and the frame for servicing, maintenance or replacement. This allows for quick lens replacement or servicing.

The frame clip may be secured to the frame via a bayonet. In particular, the frame clip and the frame may have aligned apertures through which a pin of the bayonet may be inserted. The pin of the bayonet rivets the frame clip to the frame upon rotation of the bayonet from a release position to a worn position. More particularly, the aligned apertures may be formed in the distal end portions of the frame and the frame clips. The aligned apertures may have an oblong configuration. Likewise, the pin of the bayonet may have an oblong enlarged head which corresponds to and is aligned to the oblong aligned apertures of the frame clip and frame when the bayonet is in the release position. At the release position, the enlarged head of the pin and the pin itself are insertable into the aligned apertures of the frame clip and frame. The bayonet may then be rotated to the worn position such that the oblong enlarged head of the pin is misaligned to the oblong configuration of the aligned apertures. At the worn position, the pin is not removable from the aligned apertures thereby the frame clip is secured or riveted to the frame. To further aid in the attachment of the frame clip to the frame, the upper and lower distal end portions of the frame clip may have protuberances sized and configured to be received into corresponding apertures formed in the frame. The protuberances may be sized and configured to be snapped into the corresponding apertures of the frame. Alternatively, the protuberances may have a hook so as to be insertable and hooked under the frame such that the protuberances are not removable from the frame apertures when the bayonet is attached to the frame clip and frame thereby securing the frame clip to the frame.

The sports goggle may also have soft lateral portions. In particular, the lateral distal portions of the frame may be fabricated from a softer compound or material compared to the frame itself. In particular, the frame may comprise proximal and distal walls. The middle portion of the frame may have a solid side wall that extends from the proximal wall to the distal wall. In contrast, the lateral distal portions of the frame may have a gap between the proximal end distal walls. The gap may be filled with a gap filler fabricated from a softer material compared to the frame material. The soft gap filler provides additional comfort to the wearer.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of the sports goggle with a bayonet in a worn position;

FIG. 2A is a front perspective view of the sports goggle with a left frame clip removed from a frame and a right frame clip secured to the frame;

FIG. 2B illustrates an upper distal end portion of the frame clip snapable to the frame;

FIG. 2C illustrates an upper distal end portion of the frame clip hookable under the frame;

FIG. 3 is a bottom rear perspective view of the frame illustrating a gap between proximal and distal walls of the frame to allow the lateral outer portions of the frame to flex under tension of an elastic head strap and a soft gap filler;

FIG. 4 is a right perspective view of a sports goggle illustrating the bayonet detached from the frame;

FIG. 5 is a right perspective view of the sports goggle illustrating the bayonet in a release position;

FIG. 6A is a cross-sectional view of the frame shown in FIG. 4; and

FIG. 6B is a cross-sectional view of the sports goggle shown in FIG. 1.

### DETAILED DESCRIPTION

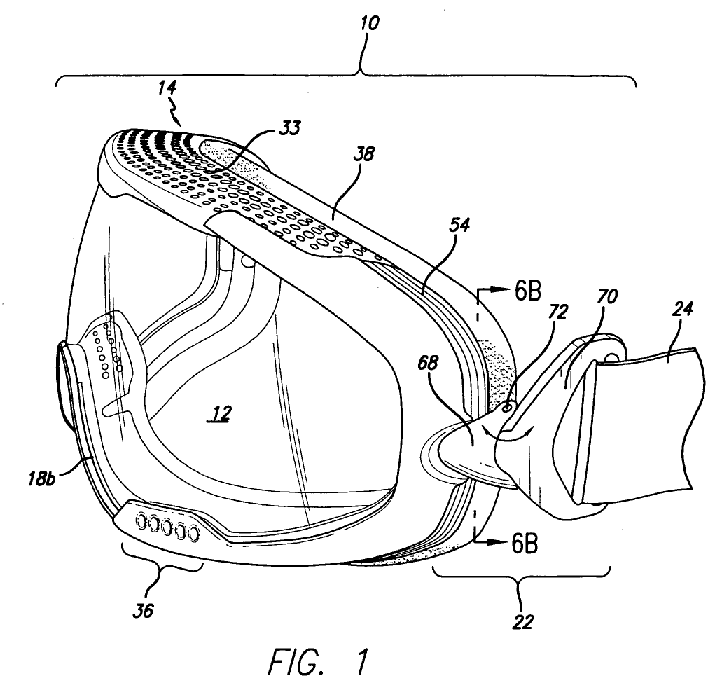

Referring now to the drawings which are for the purposes of illustration and not limitation, FIG. 1 illustrates a sports goggle 10. The sports goggle 10 discussed herein may be worn while participating in various sports such as snowboarding, skiing, motocross, moto sports, and other sport related activities. The sports goggle 10 may have a lens 12 which is removably attachable to a frame 14 of the sports goggle 10. The lens 12 may be attached to the frame 14 when the sports goggle 10 is in use. If the lens 12 is damaged or otherwise need to be replaced or maintained (e.g., cleaned), the lens 12 may be removed (as discussed herein) from the frame 14 and thrown away, replaced, cleaned or serviced. Thereafter, the lens 12 may be reattached to the frame 14 or a new lens 12 may be attached to the frame 14. Other reasons that the lens 12 may be removed from the frame 14 is to change the color of the lens 12, replacing the lens 12 with a prescription lens, or other reasons known in the art, or described herein.

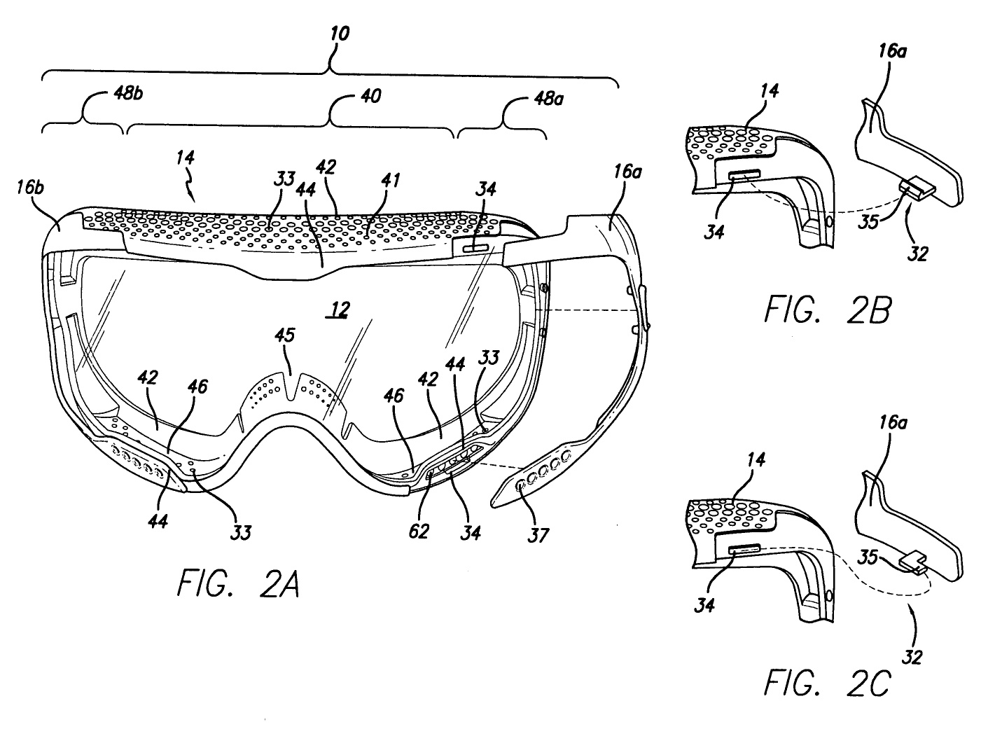

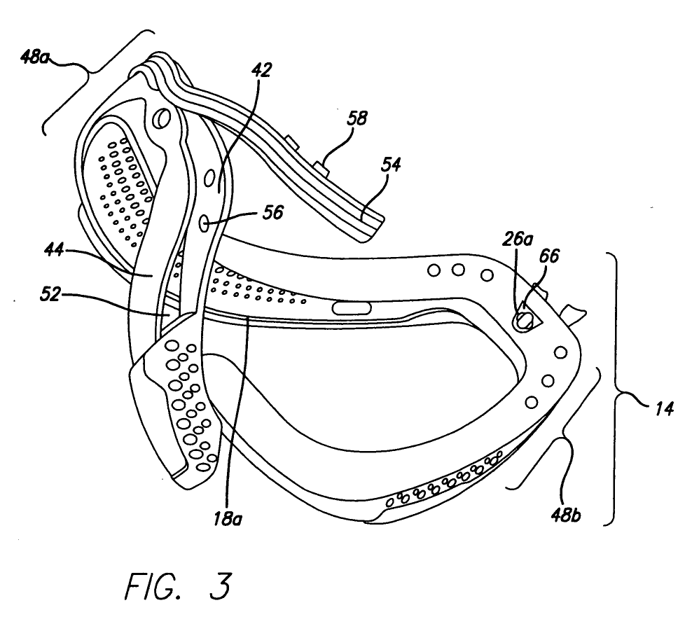

The lens 12 may be attached to the frame 14 with the aid of left and right frame clips 16a, b (see FIG. 2A). The frame clips 16a, b extends from an upper side to the side and to the lower side of the frame. Also, the lens 12 may be attached to the frame 14 with the aid of lens channels. More particularly, the lens 12 may be received into upper and lower lens channels 18a, b (see FIGS. 3 and 1). The upper channel 18a (see FIG. 3) may be formed at an upper medial portion of the frame 14. Likewise, the lower lens channel 18b (see FIG. 1) may be formed at a lower medial portion of the frame 14. The upper and lower lens channels 18a, b may be sized and configured to receive upper and lower peripheral edges of the lens 12, as shown in FIG. 1. The lens channels 18a, b may be insufficient to keep the lens 12 attached to the frame 14 during normal use of the sports goggle 10. To secure the lens 12 to the frame 14, the lens 12 may extend laterally outward to the lateral portions of the frame 14, as shown in FIG. 2A. The frame clips 16a, b may be disposed over the lateral portions of the lens 12 and attached to the frame 14 to secure the lens 12 to the frame 14. The frame clip 16a shown in FIG. 2A is detached from the frame 14. As can be seen, the left lateral portion of the lens 12 may be disposed between the frame clip 16a and the frame 14. The left frame clip 16a may then be attached to the frame to secure the left lateral portion of the lens 12 to the frame 14. Similarly, the right lateral portion of the lens 12 is disposed between the frame clip 16b and the frame 14. The frame clip 16b shown in FIG. 2A is attached to the frame 14 and secures the right lateral portion of the lens 12 to the frame 14. The left and right sides of the goggle 10 may have the same structure but be mirror images of one another. The upper and lower channels 18a, b receive the upper and lower peripheral edges of the lens 12 to hold the lens 12 to the frame 14. Also, the left and right frame clips 16a, b and the frame 14 receive the lateral peripheral edges of the lens to hold the lateral portions of the lens 12 to the frame 14. Accordingly, the entire outer periphery of the lens 12 is secured to the frame 14. It is also contemplated that the lens channels 18a, b and the left and right frame clips 16a, b secure only a portion of the outer periphery of the lens 12 to the frame 14.

To remove the lens 12 from the frame 14, the left and right frame clips 16a, b are removed from the frame 14. The lens 12 may then be removed from the lens channels 18a, b formed in the frame 14. Once the lens 12 is removed from the frame 14, the lens 12 may be cleaned or serviced and remounted to the frame 14. Alternatively, a new lens 12 may be reattached to the frame 14 with the left and right frame clips 16a, b. The lens may be a new lens without scratches or a lens with a different color, or a lens with a different structure such as a dual pane lens for reducing fogging or a prescription lens.

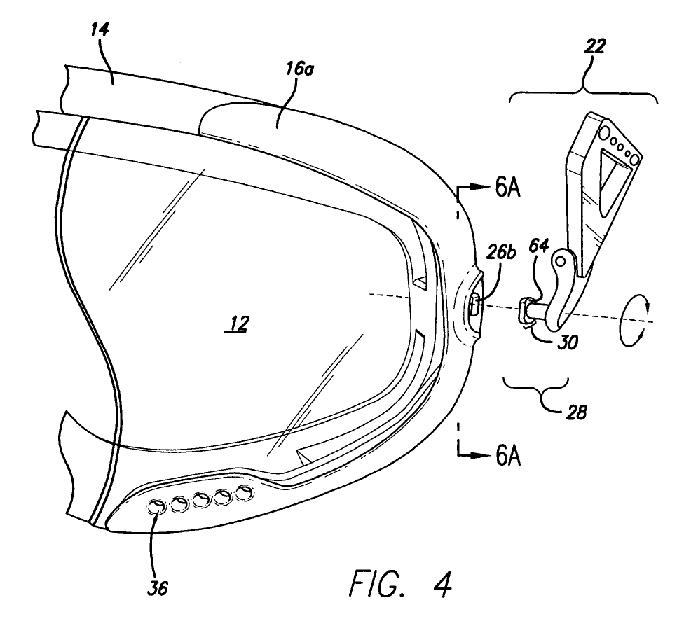

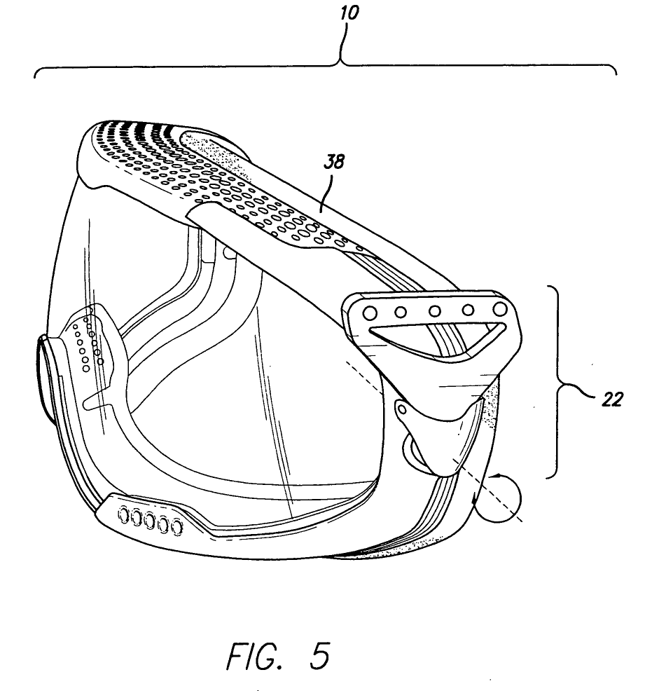

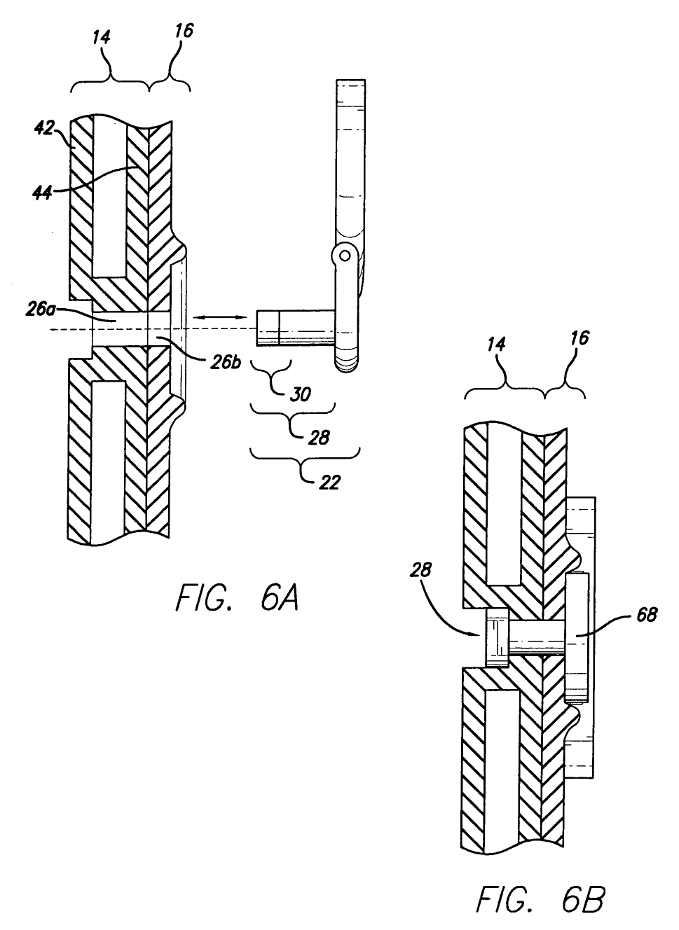

The left and right frame clips 16a, b may be secured to the frame 14 by a left bayonet 22 (see FIG. 1) and a right bayonet. The left bayonet 22 is shown and will be described for the purposes of clarity. The right bayonet may have an identical reverse structure compared to the left bayonet. The bayonet 22 may be attached to the frame 14 and an elastic head strap 24 (e.g., sonic welding), as shown in FIG. 1. The head strap 24 may be used to secure to sports goggle 10 to the head of the wearer. The bayonet 22 may be used to secure the frame clips 16a, b to the frame 14. In particular, the bayonet 22 may have a pin 28 (see FIG. 4) insertable into aligned apertures 26a, b (see FIGS. 3, 4 and 6A) of the frame 14 and frame clip 16a, b. The pin 28 may rivet the frame clip 16 to the frame 14 with the lens 12 disposed therebetween. In particular, the pin 28 may be sized and configured to be received into the aligned apertures 26a, b (see FIG. 6A) of the frame 14 and the frame clip 16. The pin 28 may have an enlarged head 30 (see FIG. 4). The enlarged head 30 of the pin 28 may have an oblong configuration (see FIG. 4). Similarly, the aligned apertures 26a, b of the frame and frame clip 16 may also have a matching oblong configuration (see FIG. 4) similar to the enlarged head 30 of the pin 28 of the bayonet 22. To attach the frame clip 16 to the frame 14, the oblong head 30 of the pin 28 may be aligned to the oblong aligned apertures 26a, b, as shown in FIGS. 4 and 6A. The pin 28 may then be inserted into the aligned apertures 26a, b, as shown in FIG. 6B. The pin 28 may have a length sufficiently long such that the enlarged head 30 may be disposed behind the frame 14, and a base 68 (see FIGS. 1 and 6B) of the bayonet 22 may be disposed in front of the frame clip 16. To ensure that the pin 28 remains disposed within the aligned apertures 26a, b, the bayonet 22 is not in the locked or worn position when the oblong, enlarged head 30 of the pin 28 is aligned to the oblong aligned apertures 26a, b. The bayonet 22 is in the release position as shown in FIG. 5. When the bayonet 22 is rotated or traversed to the worn position (see FIG. 1), the oblong shape of the enlarged head 30 of the pin 28 is misaligned to the oblong shape of the aligned apertures 26a, b, as shown in FIG. 6B. The pin 28 cannot be removed from the aligned apertures 26a, b. To unlock or remove the pin 28 from the aligned apertures 26a, b, the bayonet 22 may be rotated upwards (or downwards) until the oblong shaped enlarged head 30 is aligned to the oblong shape of the aligned apertures 26a, b, as shown in FIG. 5. Preferably, the oblong shaped enlarged head 30 is aligned to the oblong shaped aligned apertures 26a, b when the head strap attachment 22 is rotated 90° from the worn position (see FIG. 1) When the sports goggle 10 is worn by the wearer, the head strap attachment 22 is in the worn or locked position. This ensures that the pin 28 will not be removed from the aligned apertures 26a, b during normal use of the sports goggle 10.

To further secure the left and right frame clips 16a, b to the frame 14, each of the frame clips 16a, b may have an upper protuberance 32 (see FIG. 2B) and a lower protuberance rearwardly directed and located at the upper distal portion and the lower distal portion of the frame clip 16a, b. Only the upper protuberance 32 is shown for the purpose of clarity. The lower protuberance may have an identical structure and operation compared to the upper protuberance 32. The frame 14 may have corresponding apertures 34 (see FIG. 2A) for the upper and lower protuberances sized and configured to receive the protuberances 32. Preferably, the protuberances 32 may have an enlarged head (e.g., wedged, beveled, etc.) such that the protuberances 32 may be removably snapped into the apertures 34 of the frame 14, as shown in FIG. 2B.

Alternatively, the protuberances 32 may have a hook configuration, as shown in FIG. 2C. The protuberances 32 may be oriented toward the medial portion of the frame 14. To attach the frame clip 16a, b to the frame 14, the protuberances 32 are hooked into the apertures 34 of the frame 14 and under the frame 14. The head 35 of the protuberance 32 is hooked behind the frame 14. The frame clip 16a, b may then be rotated flush to the frame 14 until the aperture 26b of the frame clip 16a is aligned to the aperture 26a of the frame 14. The pin 28 of the bayonet 22 may now be inserted into the aligned apertures 26a, b.

In an aspect of the left and right frame clips 16a, b, the frame clips 16a, b may extend from an upper side of the frame 14 to a lower side of the frame 14 and be blended in with the frame 14 such that there is a smooth contoured surface between the frame 14 and the frame clips 16a, b.

The sports goggle 10 discussed herein permits a user to quickly change lens 12 for the various reasons discussed herein. In particular, the lens 12 may be changed out by rotating the bayonet 22 and removing the pin 28 from the aligned apertures 26a, b. The frame clips 26a, b may now be removed along with the lens 12. The lens 12 may be serviced or replaced. The serviced or new lens 12 may be mounted to the frame 14. Now, the pin 28 of the bayonet 22 may be inserted into the aligned apertures and the bayonet 22 rotated to the worn position.

In an aspect of the sports goggle 10, the sports goggle 10 may have air flow apertures 36 formed in a front surface of the sports goggle 10, as shown in FIG. 1. The air flow apertures 36 allow air to flow from the environment into or between the space defined by the lens 12 and the wearer's face and out through the apertures 33 (see FIG. 1) of the frame 14. The air flow apertures 36 may comprise apertures 37 (see FIG. 2A) formed in the lower distal portion of the frame clip 16. The frame 14 (see FIG. 2) may have corresponding air flow apertures 62 that are aligned to the air flow apertures 37 of the frame clip 16. During use, the sports goggle 10 is worn by the wearer. Typically, the wearer is headed downhill or into the wind. The wind will enter the air flow apertures 37, 62 of the frame clip 16 and the frame 14 and exit through apertures 33 of the frame 14. This allows the temperature of the air between the wearer's face and the lens 12 to approach the temperature of the environment (i.e., air in front of the lens 12) to mitigate fogging due to condensation on an inner surface of the lens 12. This also removes moisture from the space between the lens 12 and the wearer's face to mitigate fogging. A lower peripheral edge of the lens 12 may accommodate the apertures 37 and 62 to prevent blockage of air flow through the apertures 37, 62.

In an aspect of the sports goggle 10, the same may be more comfortable to wear due to a soft filler 54 attached to lateral portions 48a, b (see FIG. 3) of the frame 14. More particularly, the frame 14 may be an injection molded article. To position the lens 12 away from the wearer's eyes, the frame 14 may have a thickness. Additionally, a cushion 38 (see FIG. 1) may be disposed behind the frame 14 to provide a comfortable interface between the frame 14 and the wearer's face. The upper medial portion 40 (see FIG. 2A) of the frame 14 may have a solid side wall 41 extending between a proximal wall 42 and a distal wall 44. Similarly, the side walls 46 of the lower portion of the frame 14 may extend from the proximal and distal walls 42, 44 to provide a generally rigid structure. The nose portion of the frame 14 may have notches 45 (see FIG. 2A) to allow the frame 14 to conform to the shape of the wearer's face. The nose portion may be bent to match the arch of the wearer's nose. In contrast, the lateral portions 48a, b of the frame 14 may have a gap 52 (see FIG. 3) between the proximal and distal walls 42, 44. Accordingly, the lateral portions 48a, b (see FIG. 2A) of the frame 14 may be more flexible compared to the upper medial portion 40 of the frame 14 and the lower portions of the frame 14 due to the gap 52 between the proximal and distal walls 42, 44 at the lateral portions 48a, b of the frame 14.

A gap filler 54 (see FIG. 3) may be fabricated from a softer compound than the frame 14. The gap filler 54 may be sized and configured to be received into the gap 52. By way of example and not limitation, the gap filler may be fabricated from Grylamide, Acetate (Zyl/Propiunate), nickel and silver, aluminum, or other material suitable for sport goggles. Also, by way of example and not limitation, the gap filler 54 may be fabricated from silicone, rubber, plastic, pliable material, resilient material or the like. The gap filler 54 may be pre-molded into the shape of the lateral portions 48a, b (see FIGS. 2A and 3) of the frame 14 to form a smooth contour therewith, as shown in FIG. 1. The gap filler 54 may be inserted into the gap 52 and blend together with the frame clip 16. The gap filler 54 may extend from an upper side of the frame 14 around the side of the frame to a bottom side of the frame 14. Moreover, the proximal wall 42 at the lateral portions 48 of the frame 14 may have one or more apertures 56 (see FIG. 3) formed to receive protuberances 58 formed in the gap filler 54. This secures the gap filler 54 within the gap 52. Also, the gap filler 54 prevents debris from entering into the sports goggle 10 and entering into the eyes of the wearer.

In an aspect of the sports goggle 10, the lens 12 may extend to the lateral portions 48a, b of the frame 14. The lens 12 may also have pin apertures (not shown) positioned on the lens 12 so as to be alignable to the aligned apertures 26a, b when the lens 12 is received into the lens channels 18a, b. In this manner, the clip 16a, b may be placed over the lens 12. Also, the pin 28 may be inserted into the aperture 26b of the frame clip 16, the pin aperture of the lens 12 and the pin aperture 26a of the frame 14. This construction securely locks the lens 12 to the frame 14. It is contemplated that the lens 12 may be secured to the frame 14 with or without the lens channels 18a, b. Additionally or alternatively, the lens 12 may be secured to the frame with or without the lens apertures. It is further contemplated that the lens 12 may be secured to the frame 14 without frame clips 16a, b. In particular, the lens 12 may have pin apertures alignable to the apertures 26a of the frame. The pin 28 of the bayonet 22 may be inserted into the pin apertures of the lens and the apertures 26a of the frame. The pin 28 rivets the lens 12 to the frame 14.

In an aspect of the sports goggle 10, the lens 12 may have at least one aperture at the lateral portions of the lens 12. The frame 14 may, also have corresponding aperture(s) or recess(es) that is approximately the same size as the aperture(s) of the lens 12. The apertures of the lens 12 may be aligned to the apertures of the frame 14. The frame clip 16a, b may have corresponding nubs which are sized and configured to be received into the apertures of the frame and the apertures of the lens. When the frame clips 16a, b are mounted to the frame 14, the nubs are inserted into the apertures of the frame 14 and the lens 12 to prevent movement of the lens 12.

In a further aspect of the sports goggle 10, although the lens 12 is described as being held to the frame 14 with left and right frame clips 16a, b, it is also contemplated that the lens 12 may be secured to the frame 14 with only one frame clip 16a or 16b. If the left frame clip 16a solely secures the lens 12 to the frame 14, the right lateral side of the frame 14 may be slotted wherein the slot is sized and configured to receive the right peripheral edge of the lens 12. The upper and lower peripheral edges of the lens 12 may be received into the upper and lower channels 18a, b. Once the lens 12 is inserted into the lens channels 18a, b, the left frame clip 16a may be mounted to the frame 14 such that the lens 12 is disposed between the frame and the left frame clip 16a. Thereafter, the pin 28 of the bayonet 22 may be inserted into the aligned apertures 26a, b and rotated to the worn position to lock the frame clip 16a and lens 12 in position. It is contemplated that the lens 12 may be secured to the frame 14 solely with the right frame clip 16b in a similar manner discussed above in a reverse configuration.

In an aspect of the sports goggle 10, the enlarged head 30 of the pin 28 of the bayonet 22 may have a cam surface 64 (FIG. 4). The cam surface 64 of the enlarged head 30 may mate with a cam surface 66 (see FIG. 3) formed on a proximal side of the frame 14 at the lateral portions thereof. The cam surfaces 64, 66 are sized and configured to each other such that as the bayonet 22 is rotated from the release position (see FIG. 5) to the worn position (see FIG. 1), the frame clip 16, lens 12 and the frame 14 are compressed together.

In a further aspect of the sports goggle 10, as shown in FIG. 1, the bayonet 22 may comprise the base member 68 and a head strap attachment 70. The head strap attachment 70 may be rotatably attached to the base member 68 via a pin 72. When the bayonet 22 is in the worn position (FIG. 1), the head strap attachment 70 is rotatable away from the wearer's head or toward the wearer's head. This is beneficial when the sports goggle is worn in conjunction with a helmet (e.g. snowboard helmet or ski helmet). When the sports goggle 10 is worn with a helmet, the head strap 74 is routed to the exterior of the helmet. The head strap 74 does not lay flat against the side of the wearer's head, rather it is laid against the exterior side of the helmet. This forces the head strap attachment 70 to rotate outward to accommodate for the thickness of the helmet. The rotatable attachment between the head strap attachment 70 and the base member 68 accommodates the thickness of the helmet. Otherwise, the sports goggle 10 may be lifted off of the wearer's face when worn with a helmet. The rotatable attachment between the base member 68 and the head strap attachment 70 permits the user to have the advanced functionality of a sports goggle 10 that may be worn by itself or with a helmet and the ability to switch between head straps 24, frame clips 26a, b, lens 12 and frames 14.

Moreover, the head strap 24 may be rotatable with respect to the frame 14/lens 12 in two degrees of freedom. In particular, the head strap 24 may be rotated about the central axis of the pin 28 (see FIG. 4). The head strap 24 may be rotated up and down. Additionally, the head strap 24 may be rotated about the central axis of pin 72 such that the head strap 24 may accommodate the width of a helmet.

Alternatively, it is also contemplated that the base member 68 and the head strap attachment 70 may not be rotatable with respect to each other. They 68, 70 may be fixed to each other.

In an aspect of the sports goggle, the look of the sports goggle may be customizable by the dealer or retail shop. By way of example and not limitation, a retailer may be provided with a plurality of different frames 14, different lenses 12, different frame clips 16a, b and different bayonets 22. They may have different aesthetic features or prints or colors. At the retail shop, the customer or retailer may select and choose from one of the plurality of frames, one of the plurality of lenses, one of the plurality of frame clips, and one of the plurality of bayonets which the customer may mix and match to produce a style fitting for him or herself.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A sport goggle for protecting eyes of a wearer from an environment, the goggle comprising:
a frame circumscribing an eye of the wearer and defining a sight aperture through which the wearer views the environment;
a transparent lens for viewing the environment, the lens being removably attachable to the frame for servicing the lens; and
a first clip removably attachable to the frame, the first clip holding the lens adjacent the frame when the first clip is attached to the frame, and the lens being removable from the frame when the first clip is detached from the frame.

2. The goggle of claim 1 wherein the lens is disposed between the frame and the first clip.

3. The goggle of claim 1 wherein the first clip is disposed on a lateral side of the frame.

4. The goggle of claim 1 wherein the frame has a lens channel sized and configured to receive an outer peripheral edge of the lens.

5. The goggle of claim 1 further comprising a bayonet attachable to the frame for securing the first clip to the frame.

6. The goggle of claim 5 wherein the bayonet has a pin sized and configured to be received into aligned pin apertures of the first clip and the frame, the first clip being secured to the frame when the pin is received into the aligned pin apertures of the first clip and the frame and the bayonet is rotated, and the first clip being detachable from the frame when the pin is removed from the aligned pin apertures of the first clip and the frame.

7. The goggle of claim 6 wherein the bayonet is further attached to a head strap for securing the goggle against the wearer's face.

8. The goggle of claim 6 wherein the pin aperture of the frame is oblong and the pin has a matching oblong enlarged head.

9. The goggle of claim 8 wherein the oblong head of the pin is misaligned to the oblong pin aperture of the frame when the head strap is in a worn position.

10. The goggle of claim 8 wherein the frame and enlarged head of the pin have mating cam surfaces for tightening the first clip to the frame as the bayonet is traversed to a worn position.

11. The goggle of claim 1 wherein a distal portion of the first clip has a protuberance and the frame has an aperture sized and configured to receive the protuberance of the first clip.

12. The goggle of claim 10 wherein the protuberance has an enlarged distal head.

13. The goggle of claim 10 wherein the protuberance is a hook insertable into the aperture of the frame.

14. A sport goggle for protecting eyes of a wearer from an environment, the goggle comprising:
a frame circumscribing the wearer's eyes and defining a sight aperture through which the wearer views the environment;
a transparent lens for viewing the environment, the lens being removably attachable to the frame for maintaining the goggle; and
first and clips removably attachable to the frame, the first and second clips securing the lens to the frame when the first and second clips are attached to the frame, and the lens being removable from the frame when the first and second clip are detached from the frame.

15. A method of removing a lens from a frame of a sports goggle, the method comprising the steps of:
a) traversing a head strap member from a worn position to a release position, wherein a head strap of the sports goggle is routable behind a wearer's head in the worn position;
b) detaching the head strap member; and
c) removing the lens from the frame.

16. The method of claim 15 wherein the traversing step comprises the step of rotating the head strap member.

17. The method of claim 15 wherein the detaching step comprises the step of pulling a pin of the head strap member out of aligned apertures of a clip and the frame.

18. The method of claim 15 wherein the head strap member is a bayonet.

19. The method of claim 15 wherein the traversing step loosens the lens from the frame.
